(19) United States
(12) Patent Application Publication
(10) Pub. No.: US 2022/0387956 A1
(43) Pub. Date: Dec. 8, 2022

(54) MICROBALLOON PRODUCTION METHOD

(71) Applicant: TOKUYAMA CORPORATION, Yamaguchi (JP)

(72) Inventors: Yasutomo SHIMIZU, Yamaguchi (JP); Takayoshi KAWASAKI, Yamaguchi (JP)

(73) Assignee: TOKUYAMA CORPORATION, Yamaguchi (JP)

(21) Appl. No.: 17/765,655

(22) PCT Filed: Sep. 23, 2020
(86) PCT No.: PCT/JP2020/035819

§ 371 Date: Mar. 31, 2022

(30) Foreign Application Priority Data
Oct. 3, 2019 (JP) 2019-182780

(51) Int. Cl. B01J 13/16; C08G 18/73; C08G 18/32

(52) U.S. Cl. CPC B01J 13/16; C08G 18/73; C08G 18/325

(57) ABSTRACT

A method for producing microballoons consisting of a polyurethane (urea) produced by an interfacial polyaddition reaction method with a W/O emulsion and having excellent dispersibility is provided. Specifically, a method for producing microballoons, characterized by forming a microballoon dispersion liquid consisting of a polyurethane (urea) by an interfacial polyaddition reaction method with a W/O emulsion and then treating the dispersion liquid with a solution containing a monofunctional active hydrogen compound containing only one active hydrogen group selected from an amino group and a hydroxyl group is provided.

### TECHNICAL FIELD

The present invention relates to a method for producing microballoons consisting of a polyurethane (urea) having excellent dispersibility.

### BACKGROUND ART

Conventionally, microballoons encapsulating skincare ingredients, fragrance ingredients, dye ingredients, analgesic ingredients, deodorant ingredients, antioxidant ingredients, antibacterial ingredients, heat storage ingredients, and the like or hollow microballoons, the inside of which is hollow, have been used in many fields such as the fields of agrochemicals, pharmaceuticals, fragrances, liquid crystals, adhesives, electronic material parts, and building materials.

As a method for producing such microballoons, a coacervation method, an in-situ polymerization method, an interfacial polyaddition reaction method, and the like are known. Of these, the interfacial polyaddition reaction method is a method in which in the case of, for example, polyurethane, polyurea, or polyurethane urea (hereinafter collectively referred to as “polyurethane (urea)”), a polyurea resin film, a polyurethane resin film, or a polyurethane urea resin film is formed by reacting mainly a polyisocyanate compound with water, polyamine, polyhydric alcohol, or amino alcohol. Specifically, it is a method in which both the continuous phase and the dispersed phase dispersed therein are allowed to contain different types of monomers to be emulsified, and a resin film is formed at the interface therebetween, which means the surface of the dispersed phase.

As this interfacial polyaddition reaction method, a method of microballooning in which an oil-in-water (O/W) emulsion in which the continuous phase is water and the dispersed phase is oil, or water in oil (W/O) emulsion in which the continuous phase is oil and the dispersed phase is water (hereinafter, also referred to as “W/O emulsion”) is known.

In Patent Document 1, a microballoon dispersion liquid, in which a hydrophobic solvent contains microballoons consisting of capsules of encapsulated polyurethane and/or polyurea that are prepared by an interfacial polycondensation method with a W/O emulsion having a dispersed phase of a water-soluble organic substance, is proposed.

The method described in Patent Document 1 can produce a microballoon dispersion liquid. However, according to the study by the present inventors, it was found that when the microballoons are isolated from the microballoon dispersion liquid, the aggregation of microballoons occurs, probably due to residual unreacted isocyanate groups, which is undesirable for subsequent handling.

Further, for the purpose of reducing the viscosity of the microballoon dispersion liquid, Patent Document 2 proposes a method for suppressing the thickening of a microballoon dispersion liquid by reacting an isocyanate with a compound having isocyanate-reactive groups by an interfacial polyaddition method using a dispersed phase containing a water-soluble organic substance to form a microballoon dispersion liquid, and then post-treating the microballoon dispersion liquid with a compound selected from amines, alcohols, and amino alcohols, such as animated fatty alcohol having a molecular weight of at least 150 g/mol.

### PRIOR ART DOCUMENTS

### Patent Document

Patent Document: Japanese Unexamined Patent Publication No. 2004-538354

Patent Document: Japanese Unexamined Patent Publication No. 2008-518765

### SUMMARY OF THE INVENTION

### Technical Problem

The method described in Patent Document 2 can more effectively suppress the thickening of the microballoon dispersion liquid. However, although the method described in Patent Document 2 can produce a low viscosity microballoon dispersion liquid, there is room for improvement in the aggregation of microballoons when the microballoons are isolated, and the cost itself has not been satisfactory.

Therefore, an object of the present invention is to provide a microballoon consisting of a polyurethane (urea) which is produced by an interfacial polyaddition reaction method using a W/O emulsion, the microballoon having excellent dispersibility.

### Solution to Problem

As a result of intensive studies to achieve the objective described above, the present inventors found that the object can be achieved by forming a microballoon dispersion liquid consisting of a polyurethane (urea) by an interfacial polyaddition reaction method using a W/O emulsion and then treating the dispersion liquid with a solution containing a monofunctional active hydrogen compound containing only one active hydrogen group selected from an amino group and a hydroxyl group. This has led to the completion of the present invention.

In other words, the present invention relates to:

a method for producing microballoons, comprising: mixing and stirring (a) an organic solvent solution containing surfactant and (b) an aqueous solution containing at least one active hydrogen group-containing compound selected from the group consisting of a polyol, a polyamine, and a compound having both hydroxyl and amino groups to prepare a W/O emulsion in which the organic solvent solution is a continuous phase, and the aqueous solution is a dispersed phase; adding (c) a polyfunctional isocyanate compound having at least two isocyanate groups to the W/O emulsion to react the polyfunctional isocyanate compound and the active hydrogen group-containing compound on the interface of the W/O emulsion to form microballoons consisting of a polyurethane (urea) to obtain a microballoon dispersion liquid in which the formed microballoons are dispersed; and treating the microballoons in a solution containing (d) a monofunctional active hydrogen compound containing only one active hydrogen group selected from an amino group and a hydroxyl group after forming the microballoons.

### Effects of the Invention

The microballoons obtained by the method of the present invention are characterized in that they do not aggregate even when isolated and show excellent dispersibility. Furthermore, as the microballoons can encapsulate a water-soluble compound in the dispersed phase inside thereof, they can be functional microballoons encapsulating skincare ingredients, fragrance ingredients, dye ingredients, analgesic ingredients, deodorant ingredients, antioxidant ingredients, antibacterial ingredients, heat storage ingredients, and the like or hollow microballoons, the inside of which is hollow, and thus they can be used in many fields such as the fields of agrochemicals, pharmaceuticals, fragrances, liquid crystals, adhesives, electronic material parts, and building materials.

Further, in the application of blending the microballoons with a resin consisting of a polyurethane (urea), since the same resin is used, the dispersibility and compatibility with the resin consisting of a polyurethane (urea) are excellent.

Therefore, in particular, they can be expected to be applied to a polishing pad for chemical mechanical polishing (CMP) made of a polyurethane (urea) used for wafer polishing.

Hollow microballoons are used in the polishing pad for CMP to provide pores. Conventionally, microballoons of a vinylidene chloride resin or the like with inorganic particles sprinkled on the surface thereof have been known to improve dispersibility in a polyurethane (urea), while inorganic particles may cause defects on wafers.

However, since the microballoons obtained by the method of the present invention have excellent dispersibility and compatibility without being sprinkled with inorganic particles or the like, it is possible to make a polishing pad with reduced defects.

### DESCRIPTION OF THE EMBODIMENTS

The method for producing microballoons of the present invention is divided into: a first step: a step of preparing (a) an organic solvent solution containing a surfactant (hereinafter, also referred to as “component (a)”); a second step: a step of preparing (b) an aqueous solution containing at least one active hydrogen group-containing compound selected from the group consisting of a polyol, a polyamine, and a compound having both hydroxyl and amino groups (hereinafter, also referred to as “component (b)”); a third step: a step of preparing a W/O emulsion in which the organic solvent solution is a continuous phase and the aqueous solution is a dispersed phase by mixing and stirring (a) and (b) described above; a fourth step: a step of obtaining a microballoon dispersion liquid in which the formed microballoons are dispersed by adding (c) a polyfunctional isocyanate compound having at least two isocyanate groups (hereinafter, also referred to as “component (c)”) to the W/O emulsion to react the polyfunctional isocyanate compound and the active hydrogen group-containing compound on the interface of the W/O emulsion so as to form microballoons consisting of a polyurethane (urea); and a fifth step: a step of treating the microballoons in a solution containing (d) a monofunctional active hydrogen compound containing only one active hydrogen group selected from an amino group and a hydroxyl group (hereinafter, also referred to as “component (d)”) after forming the microballoons. In the present invention, the obtained microballoons can be used in a state of containing the aqueous solution inside the microballoons or as hollow microballoons after removing the aqueous solution depending on the application. The above-described first and second steps can also be carried out in the reverse order for production. The particle diameter of the microballoons that can be used in the present invention is preferably from 1 μm to 200 μm, and the average particle diameter thereof is more preferably from 10 μm to 100 μm. A known method can be used for measuring the average particle diameter. Specifically, an image analysis method can be used. The particle diameter can be easily measured by using the image analysis method. The average particle diameter is the average particle diameter of the primary particles.

In the present invention, the term “W/O emulsion” or “water-in-oil (W/O) emulsion” refers to a macroscopically homogeneous composition, which is an emulsion containing a continuous oil phase (continuous phase) and an aqueous phase (dispersed phase) in the form of droplets dispersed in the oil phase.

Hereinafter, the method for producing the microballoon of the present invention will be described.

### (Method for Producing Microballoons Consisting of Polyurethane (Urea)) First Step:

The first step is a step of preparing (a) an organic solvent solution containing a surfactant which becomes a continuous phase in a W/O emulsion.

This step is a step of dissolving a surfactant described later in an organic solvent described later to prepare an organic solvent solution, and a uniform solution can be obtained via dissolution by a known method.

In the present invention, the amount of the surfactant used is usually from 0.01 to 10 parts by mass, preferably from 0.1 to 10 parts by mass with respect to 100 parts by mass of the organic solvent. Within this range, the aggregation of droplets of the dispersed phase in the W/O emulsion is avoided, and it is easy to obtain microballoons having a uniform average particle diameter.

Further, an urethanization catalyst described later can be added to the component (a) for the purpose of accelerating the reaction between an isocyanate compound described later and a polyol, a polyamine, and a compound having both hydroxyl and amino groups.

### Second Step:

The second step is a step of preparing (b) an aqueous solution containing at least one of a polyol, a polyamine, and an active hydrogen-containing compound having both hydroxyl and amino groups, which becomes a dispersed phase in the W/O emulsion.

This step is a step of dissolving at least one of a polyol, a polyamine, and an active hydrogen-containing compound having both hydroxyl and amino groups, which will be described later, in water to prepare an aqueous solution, and a uniform solution can be obtained via dissolution by a known method.

The amount of at least one active hydrogen-containing compound having polyol, the polyamine, and both a hydroxyl group and an amino group used in the present invention is usually from 0.5 to 50 parts by mass, preferably from 1 to 30 parts by mass, more preferably from 2 to 20 parts by mass with respect to 100 parts by mass of water. Within this range, by producing a W/O emulsion, it is easy to produce a polyurethane (urea) resin film, thereby making it possible to obtain favorable microballoons.

In addition, the component (b) used in the present invention can contain a water-soluble compound for the purpose of imparting functionality to the microballoons. In that case, the amount of the water-soluble compound added is generally in a range of from 1 to 50 parts by mass with respect to 100 parts by mass of the component (b). In this case, microballoons encapsulating the contained water-soluble compound can be obtained.

Further, an urethanization catalyst described later can be added to the component (b) for the purpose of accelerating the reaction between an isocyanate compound described later and a polyol, a polyamine, and/or a compound having both hydroxyl and amino groups.

### Third Step:

The third step is a step of mixing and stirring the component (a) obtained in the first step and the component (b) obtained in the second step to prepare a W/O emulsion in which the component (a) is a continuous phase, and the component (b) is a dispersed phase.

In the present invention, the method for mixing and stirring the component (a) and the component (b) to form a W/O emulsion is to mix and stir appropriately by a known method in consideration of the particle diameter of a microballoon to be produced, thereby allowing the preparation of the W/O emulsion. The particle diameter of the W/O emulsion substantially corresponds to the size of the particle diameter of the obtained microballoons.

In particular, the method of W/O emulsification by a method for mixing the component (a) and the component (b) and then dispersing them using a known disperser such as a high-speed shear-, friction-, high-pressure jet-, or ultrasonic-disperser for stirring is preferably adopted, and a high-speed shear-disperser is preferable. In a case in which a high-speed shear-disperser is used, the rotation speed is preferably from 1,000 to 20,000 rpm, more preferably from 1,500 to 10,000 rpm. The dispersion time is preferably from 0.1 to 60 minutes, more preferably from 0.5 to 30 minutes. The dispersion temperature is preferably from 10° C. to 40° C.

Further, in the present invention, the weight ratio of the component (a) and the component (b) is that the component (b) is preferably from 1 to 100 parts by mass, more preferably from 5 to 90 parts by mass, most preferably from 10 to 80 parts by mass when the component (a) is 100 parts by mass. Within this range, a favorable emulsion can be obtained.

### Fourth Step:

The fourth step is a step of adding (c) a polyfunctional isocyanate compound having at least two isocyanate groups to the W/O emulsion to react the polyfunctional isocyanate compound and the active hydrogen group-containing compound on the interface of the W/O emulsion to form microballoons consisting of a polyurethane (urea) resin film so as to obtain a microballoon dispersion liquid in which the formed microballoons are dispersed.

The amount of the component (c) used in the present invention which will be described later is preferably from 5 to 500 parts by mass, more preferably from 10 to 300 parts by mass, most preferably from 30 to 200 parts by mass with respect to 100 parts by mass of the polyol, polyamine, or active hydrogen-containing compound having both hydroxyl and amino groups. Within this range, an excellent resin film can be formed.

Moreover, the component (c) can be used as it is or can be used by dissolving it in the above-described organic solvent. When using an organic solvent, it is preferable that the same organic solvent as that used for the component (a) is used.

In a case in which the above-described organic solvent is used, it is preferable to use the organic solvent in a range of from 50 to 1000 parts by mass with respect to 100 parts by mass of the component (c).

The reaction temperature is not particularly limited as long as the W/O emulsion is not broken, and the reaction is carried out preferably in a range of from 5° C. to 70° C. The reaction time is not particularly limited as long as the W/O emulsion can be formed, and it is usually selected from a range of from 1 to 480 minutes.

### Fifth Step:

The fifth step is a step of treating the microballoons consisting of a polyurethane (urea) in a solution containing (d) a monofunctional active hydrogen compound containing only one active hydrogen group selected from an amino group and a hydroxyl group, which is described later.

According to the study by the present inventors, it was found that in a case in which microballoons are separated and dried from the microballoon dispersion liquid obtained by the interfacial polyaddition reaction, the remaining isocyanate group may cause the aggregation of the microballoons. In the present invention, the microballoons are treated in a solution containing the component (d) to form a urethane bond or a urea bond of the residual isocyanate group such that microballoons having excellent dispersibility can be obtained.

The method for treating the microballoons with a solution containing the component (d) is not particularly limited, and the following methods are preferably used.

(1) A method in which the microballoons are once separated from the microballoon dispersion liquid, the separated microballoons are dispersed in a solution containing the component (d), and then the microballoons are re-separated.
(2) A method in which the microballoon dispersion liquid and a solution containing the component (d) are mixed, and then the microballoons are separated from the microballoon dispersion liquid.

Among them, the method (1) is suitable because the dispersibility of the obtained microballoons becomes better. The details will be described below.

The solution containing the component (d) used in the present invention can be a solution containing only the component (d) when the component (d) is a liquid and can be a mixed solution of the component (d) with a different solvent as long as the effects of the present invention are not impaired. When the component (d) is a solid, it is preferable to dissolve it in a different solvent to prepare a solution containing the component (d). The different solvent can be used without any particular limitation as long as it is inert to isocyanate groups and it can be miscible with the component (d).

Further, the amount of the component (d) used in the present invention can be adjusted depending on the amount of the component (c), and it is preferably from 0.1 to 20 parts by mass, more preferably from 0.2 to 15 parts by mass, most preferably from 0.5 to 10 parts by mass with respect to 1 part by mass of the component (c). Within this range, the dispersion between the microballoons is favorable.

Further, a urethanization catalyst described later can be added to the solution containing the component (d) for the purpose of accelerating the reaction between isocyanate groups and the component (d).

In the method (1) described above, the separation method for separating the microballoons from the microballoon dispersion liquid can be selected from general separation methods without particular limitation, and specifically, filtration, centrifugation, or the like can be used.

Further, the method for drying the obtained microballoons can be selected from known methods, and for example, the microballoons can be dried in a circulation dryer in a range of from 40° C. to 150° C. When removing the aqueous solution from the microballoons to form hollow microballoons, vacuum drying can be performed to remove the aqueous solution inside if necessary.

Further, a known method can be adopted without particular limitation as the method for dispersing the microballoons in the solution containing the component (d), and the above-described separation method can be adopted without particular limitation as the method of re-separating the microballoons.

In particular, a method in which the microballoons are once separated from the above-described microballoon dispersion liquid, the microballoons are dispersed in a solution containing the component (d) without drying, and then the microballoons are re-separated is preferably used because of the dispersibility of the obtained microballoons.

Known methods can be adopted without particular limitation as a method in which the microballoon dispersion liquid and a solution containing the component (d) are mixed and a method in which the microballoons are separated from the microballoon dispersion liquid following the mixing in the method (2) described above.

Each component used in the present invention will be described below.

### <Surfactant>

In the present invention, as the surfactant used for the component (a), a known surfactant can be used without any limitation as long as it is soluble in an organic solvent described later.

Examples of the surfactant include anionic surfactants, cationic surfactants, amphoteric surfactants, and nonionic surfactants. The surfactant can be a combination of two or more kinds of surfactants.

Examples of anionic surfactants include carboxylic acids or salts thereof, sulfate salts, salts of carboxymethylated products, sulfonates, and phosphate salts.

Examples of carboxylic acids or salts thereof include saturated or unsaturated fatty acids having carbon number of 8 to 22 or salts thereof, which are specifically capric acid, lauric acid, myristic acid, palmitic acid, stearic acid, arachidic acid, behenic acid, oleic acid, linolenic acid, linoleic acid, ricinoleic acid, and mixtures of higher fatty acids obtained by saponifying palm oil, palm kernel oil, rice bran oil, beef tallow, and the like. Examples of the salts include salts of sodium, potassium, ammonium, alkanolamine, and the like.

Examples of sulfate salts include higher alcohol sulfate salts (sulfate salts of fatty alcohols having carbon number of 8 to 18), higher alkyl ether sulfate salts (sulfate salts of ethylene oxide adducts of fatty alcohols having carbon number of 8 to 18), sulfated oils (products obtained by directly sulfating and neutralizing unsaturated fats and oils or unsaturated waxes), sulfated fatty acid esters (products obtained by sulfating and neutralizing lower alcohol esters of unsaturated fatty acids), and sulfated olefins (products obtained by sulfating and neutralizing olefins having carbon number of 12 to 18). Examples of the salts include sodium salts, potassium salts, ammonium salts, and alkanolamine salts.

Specific examples of higher alcohol sulfate salts include octyl alcohol sulfate salts, decyl alcohol sulfate salts, lauryl alcohol sulfate salts, stearyl alcohol sulfate salts, and sulfate salts of alcohols synthesized by the oxo method (OXOCOL 900, tridecanol manufactured by KYOWA HAKKO BIO CO., LTD.).

Specific examples of higher alkyl ether sulfate salts include lauryl alcohol ethylene oxide 2-mol adduct sulfate salts and octyl alcohol ethylene oxide 3-mol adduct sulfate salts.

Specific examples of sulfated oils include sodium, potassium, ammonium, and alkanolamine salts of sulfated products of castor oil, peanut oil, olive oil, rapeseed oil, beef tallow, sheep tallow, and the like.

Specific examples of sulfated fatty acid esters include sodium, potassium, ammonium, and alkanolamine salts of sulfated products of butyl oleate, butyl ricinoleate, and the like.

Examples of salts of carboxymethylated products include salts of carboxymethylated products of fatty alcohols having carbon number of 8 to 16 and salts of carboxymethylated products of fatty alcohol ethylene oxide adducts having carbon number of 8 to 16.

Specific examples of salts of carboxymethylated products of fatty alcohols include octyl alcohol carboxymethylated sodium salts, decyl alcohol carboxymethylated sodium salts, lauryl alcohol carboxymethylated sodium salts, and tridecanol carboxymethylated sodium salts.

Specific examples of salts of carboxymethylated products of fatty alcohol ethylene oxide adducts include octyl alcohol ethylene oxide 3-mol adduct carboxymethylated sodium salts, lauryl alcohol ethylene oxide 4-mol adduct carboxymethylated sodium salts, and tridecanol ethylene oxide 5-mol adduct carboxymethylated sodium salts.

Examples of sulfonates include alkylbenzene sulfonates, alkylnaphthalene sulfonates, sulfosuccinic acid diester sulfonates, α-olefin sulfonates, Igepon T sulfonates, and sulfonates of other aromatic ring-containing compounds.

Specific examples of alkylbenzene sulfonates include dodecylbenzene sulfonic acid sodium salts.

Specific examples of alkylnaphthalene sulfonate include dodecyl naphthalene sulfonic acid sodium salts.

Specific examples of sulfosuccinic acid diester sulfonates include sulfosuccinic acid di-2-ethylhexyl ester sodium salts.

Examples of sulfonates of aromatic ring-containing compounds include mono or disulfonates of alkylated diphenyl ether and styrenated phenol sulfonates.

Examples of phosphate salts include higher alcohol phosphate salts and higher alcohol ethylene oxide adduct phosphate salts.

Specific examples of higher alcohol phosphate salts include lauryl alcohol phosphoric acid monoester disodium salts and lauryl alcohol phosphoric acid diester sodium salts.

Specific examples of higher alcohol ethylene oxide adduct phosphate salts include oleyl alcohol ethylene oxide 5-mol adduct phosphoric acid monoester disodium salts.

Examples of cationic surfactants include quaternary ammonium salt surfactants and amine salt surfactants.

Quaternary ammonium salt surfactants can be obtained by reacting tertiary amines and quaternization agents (alkylating agents such as methyl chloride, methyl bromide, ethyl chloride, benzyl chloride, and dimethyl sulfate, ethylene oxide, and the like). Examples thereof include lauryl trimethyl ammonium chloride, didecyldimethylammonium chloride, dioctyldimethylammonium bromide, stearyl trimethylammonium bromide, lauryldimethylbenzylammonium chloride (benzalkonium chloride), cetylpyridinium chloride, polyoxyethylene trimethylammonium chloride, and stearamide ethyl diethyl methyl ammonium methosulfate.

Amine salt surfactants can be obtained by neutralizing primary to tertiary amines with inorganic salts (e.g., hydrochloric acid, nitric acid, sulfuric acid, and hydroiodic acid) or organic acids (e.g., acetic acid, formic acid, oxalic acid, lactic acid, gluconic acid, adipic acid, and alkylphosphoric acid). For example, examples of primary amine salt surfactants include inorganic acid salts or organic acid salts of higher aliphatic amines (higher amines such as laurylamine, stearylamine, cetylamine, hardened beef tallow amine, and rosin amine), higher fatty acid (e.g., stearic acid, oleic acid) salts of lower amines.

Examples of secondary amine salt surfactants include inorganic or organic acid salts such as, for example, ethylene oxide adducts of aliphatic amines.

In addition, examples of tertiary amine salt surfactants include: inorganic acids salts or organic acid salts of aliphatic amines (e.g., triethylamine, ethyldimethylamine, and N,N,N′,N′-tetramethylethylenediamine), ethylene oxide adducts of aliphatic amines, alicyclic amines (e.g., N-methylpyrrolidine, N-methylpiperidine, N-methylhexamethyleneimine, N-methylmorpholine, and 1,8-diazabicyclo(5,4,0)-7-undecene), nitrogen-containing heterocyclic aromatic amines (e.g., 4-dimethylaminopyridine, N-methylimidazole, and 4,4′-dipyridyl); and inorganic acids salts or organic acid salts of tertiary amines such as triethanolamine monostearate and stearamide ethyldimethyl methylethanolamine.

Examples of amphoteric surfactants include carboxylate amphoteric surfactants, sulfate salt amphoteric surfactants, sulfonate amphoteric surfactants, and phosphate salt amphoteric surfactants. Further, examples of carboxylate amphoteric surfactants include amino acid amphoteric surfactants and betaine amphoteric surfactants.

Examples of carboxylate amphoteric surfactants include amino acid amphoteric surfactants, betaine amphoteric surfactants, and imidazoline amphoteric surfactants. Of these, amino acid amphoteric surfactants are amphoteric surfactants each having an amino group and a carboxyl group in the molecule. Specific examples thereof include alkylaminopropionic acid amphoteric surfactants (e.g., sodium stearyl aminopropionate and sodium lauryl aminopropionate), and alkylaminoacetic acid amphoteric surfactants (e.g., sodium laurylaminoacetate).

Betaine amphoteric surfactants are amphoteric surfactants each having a quaternary ammonium salt cation moiety and a carboxylic acid anion moiety in the molecule, which include, for example, alkyldimethylbetaine (e.g., stearyl dimethylaminoacetic acid betaine and betaine lauryldimethylaminoacetic acid), amide betaine (e.g., coconut oil fatty acid amide propyl betaine), and alkyl dihydroxyalkyl betaine (e.g., lauryl dihydroxyethyl betaine).

Further, examples of imidazoline amphoteric surfactants include 2-undecyl-N-carboxymethyl-N-hydroxyethylimidazolinium betaine.

Examples of other amphoteric surfactants include: glycine amphoteric surfactants such as sodium lauroyl glycine, sodium lauryl diaminoethyl glycine, lauryl diaminoethylglycine hydrochloride, and dioctyldiaminoethylglycine hydrochloride; and sulfobetaine amphoteric surfactants such as pentadecyl sulfotaurine.

Examples of nonionic surfactants include alkylene oxide adduct nonionic surfactants and polyhydric alcohol nonionic surfactants.

An alkylene oxide adduct nonionic surfactant can be obtained by adding alkylene oxide directly to higher alcohol, higher fatty acid, alkylamine, or the like, by reacting higher fatty acid or the like with a polyalkylene glycol obtained by adding alkylene oxide to a glycol, by adding alkylene oxide to an esterified product obtained by reacting polyhydric alcohol with higher fatty acid, or by adding alkylene oxide to higher fatty acid amide.

Examples of alkylene oxide include ethylene oxide, propylene oxide, and butylene oxide.

Specific examples of alkylene oxide adduct nonionic surfactants include: oxyalkylene alkyl ethers (e.g., octyl alcohol ethylene oxide adducts, lauryl alcohol ethylene oxide adducts, stearyl alcohol ethylene oxide adducts, oleyl alcohol ethylene oxide adducts, and lauryl alcohol ethylene oxide propylene oxide block adducts); polyoxyalkylene higher fatty acid esters (e.g., stearyl acid ethylene oxide adducts and lauryl acid ethylene oxide adducts); polyoxyalkylene polyhydric alcohol higher fatty acid esters (e.g., polyethylene glycol laurate diester, polyethylene glycol oleate diester, and polyethylene glycol stearate diester); polyoxyalkylene alkyl phenyl ethers (e.g., nonylphenol ethylene oxide adducts, nonylphenol ethylene oxide propylene oxide block adducts, octylphenol ethylene oxide adducts, bisphenol A ethylene oxide adducts, dinonylphenol ethylene oxide adducts, and styrenated phenol ethylene oxide adducts); polyoxyalkylene alkyl amino ethers (e.g., laurylamine ethylene oxide adducts and stearylamine ethylene oxide adducts); and polyoxyalkylene alkyl alkanolamides (e.g., ethylene oxide adducts of hydroxyethyllauric acid amide, ethylene oxide adducts of hydroxypropyl oleic acid amide, and ethylene oxide adducts of dihydroxyethyl lauric acid amide).

Examples of polyhydric alcohol nonionic surfactants include polyhydric alcohol fatty acid esters, polyhydric alcohol fatty acid ester alkylene oxide adducts, polyhydric alcohol alkyl ethers, and polyhydric alcohol alkyl ether alkylene oxide adducts.

Specific examples of polyhydric alcohol fatty acid esters include pentaerythritol monolaurate, pentaerythritol monooleate, sorbitan monolaurate, sorbitan monostearate, sorbitan monolaurate, sorbitan dilaurate, sorbitan dioleate, and sucrose monostearate.

Specific examples of polyhydric alcohol fatty acid ester alkylene oxide adducts include ethylene glycol monooleate ethylene oxide adducts, ethylene glycol monostearate ethylene oxide adducts, trimethylolpropane monostearate ethylene oxide propylene oxide random adducts, sorbitan monolaurate ethylene oxide adducts, sorbitan monostearate ethylene oxide adducts, sorbitan distearate ethylene oxide adducts, and sorbitan dilaurate ethylene oxide propylene oxide random adducts.

Specific examples of polyhydric alcohol alkyl ethers include pentaerythritol monobutyl ether, pentaerythritol monolauryl ether, sorbitan monomethyl ether, sorbitan monostearyl ether, methyl glycoside, and lauryl glycoside.

Specific examples of polyhydric alcohol alkyl ether alkylene oxide adducts include sorbitan monostearyl ether ethylene oxide adducts, methyl glycoside ethylene oxide propylene oxide random adducts, lauryl glycoside ethylene oxide adducts, and stearyl glycoside ethylene oxide propylene oxide random adducts.

Among them, the surfactant used in the present invention is preferably selected from nonionic surfactants, it is more preferably selected from polyhydric alcohol fatty acid esters among nonionic surfactants, and it is most preferably cyclized sorbitol.

Specific examples of the most preferable surfactants include sorbitan monostearate (trade name: span (registered trademark) 60), sorbitan monooleate (trade name: span (registered trademark) 80), sorbitan trioleate (trade name: span (registered trademark) 85).

### <Organic Solvent>

In the present invention, as an organic solvent used for the component (a), a known organic solvent that is incompatible with water can be used without any limitation.

As the organic solvent, those generally known as hydrophobic solvents, hydrocarbon oils, ester oils, and ether oils can be used. A preferable hydrophobic solvent used in the present invention is one having a solubility in water of 1 g/L or less at 25° C.

Examples of a hydrophobic solvent include aliphatic solvents such as C6-C12-hydrocarbons, which are particularly n-hexane, n-heptane, n-octane, and cyclohexane, aromatic solvents such as benzene, toluene, and xylene, and halogenated solvents which are generally chlorides such as chloroform, dichloromethane, tetrachloromethane, and mono or dichlorobenzene.

Other examples include hydrocarbon oils, ester oils, ether oils, higher fatty acids, and animal and vegetable oils. Examples thereof include: hydrocarbon oils such as liquid paraffin, liquid isoparaffin, hydrogenated polyisobutene, squalane, and n-hexadecane; ester oils such as diisostearyl malate, octyldodecyl lactat, isotridecyl isononanoate, octyldodecyl myristate, isopropyl palmitate, isopropyl isostearate, butyl stearate, myristyl myristate, isopropyl myristate, octyldodecyl myristate, di(2-ethylhexyl) adipate, diisopropyl sebacate, neopentyl glycol dicaprate, and tricaproin; ether oils such as dioctyl ether, ethylene glycol monolauryl ether, ethylene glycol dioctyl ether, and glycerol monooleyl ether; higher fatty acids such as capric acid, lauric acid, myristic acid, palmitic acid, stearic acid, arachidic acid, behenic acid, oleic acid, linolenic acid, linoleic acid, ricinoleic acid; and animal and vegetable oils such as camellia oil, soybean oil, corn oil, cottonseed oil, rapeseed oil, olive oil, palm oil, castor oil, and fish oil.

These solvents can be used singly or as a mixed solvent of two or more kinds thereof.

The organic solvent used in the present invention is preferably n-hexane, toluene, a hydrocarbon oil, a higher fatty acid, an animal or vegetable oil, or the like, and a higher fatty acid or an animal or vegetable oil is particularly preferable. It becomes easy to produce a stable emulsion by using them.

<(b) At Least One Active Hydrogen Group-Containing Compound Selected from the Group Consisting of Polyol, Polyamine, and Compound Having both Hydroxyl and Amino Groups>

The polyol, polyamine, or compound having both hydroxyl and amino groups used in the present invention can be used without limitation as long as it is a water-soluble compound containing at least two active hydrogens.

In the present invention, the water-soluble compound is a compound that is at least partially soluble in water and has a higher affinity in a hydrophilic phase than in a hydrophobic phase. In general, a compound having a solubility of at least 1 g/L in a hydrophilic solvent such as water at room temperature can be selected. Preferably, a water-soluble compound having a solubility of ≥20 g/L in a hydrophilic solvent at 25° C. can be mentioned.

Specific examples of such a water-soluble compound which is a polyol, a polyamine, or a compound having both hydroxyl and amino groups and containing at least two active hydrogens are shown below.

A water-soluble polyol is a polyfunctional alcohol having two or more hydroxyl groups in the molecule. Specific examples thereof include: bifunctional polyols such as ethylene glycol, diethylene glycol, triethylene glycol, polyethylene glycol, propylene glycol, dipropylene glycol, tripropylene glycol, polypropylene glycol, neopentyl glycol, 1,2-butanediol, 1,3-butanediol, 2,3-butanediol, 1,4-butanediol, 1,5-pentanediol, hexylene glycol, 1,6-hexanediol, and 2-butene-1,4-diol; trifunctional polyols such as glycerin, trimethylolethane, and trimethylolpropane; tetrafunctional polyols such as pentaerythritol, erythritol, diglycerol, diglycerin, and ditrimethylolpropane; pentafunctional polyols such as arabitol; hexafunctional polyols such as dulcitol, sorbitol, mannitol, dipentaerythritol, and triglycerol; heptafunctional polyols such as volemitol; nonafunctional polyols such as isomalt, maltitol, isomaltitol, and lactitol; and water-soluble polymers such as cellulose compounds (e.g., methyl cellulose, ethyl cellulose, hydroxyethyl cellulose, ethyl hydroxyethyl cellulose, carboxymethyl cellulose, hydroxypropyl cellulose, and saponified products thereof), starch, dextrin, cyclic dextrin, chitin, polyvinyl alcohol, and polyglycerin.

A water-soluble polyamine is a polyfunctional amine having two or more amino groups in the molecule. Specific examples thereof include ethylenediamine, propylenediamine, 1,4-diaminobutane, hexamethylenediamine, 1,8-diaminooctane, 1,10-diaminodecane, dipropylene triamine, bis(hexamethylene) triamine, tris(2-aminoethyl)amine, tris(3-aminopropyl) amine, 3,3′,3″-nitrilotris(propionamide), piperazine, 2-methylpiperazine, isophoronediamine, diethylenetriamine, triethylenetetramine, tetraethylenepentamine, hydrazine, polyethyleneimines, and polyoxyalkylene amines.

A compound having both a water-soluble hydroxyl group and an amino group is a polyfunctional water-soluble compound having two or more hydroxyl and amino groups in total in the molecule. Specific examples thereof include hydroxylamine, monoethanolamine, 3-amino-1-propanol, 2-amino-2-hydroxymethylpropane-1,3-diol, 2-hydroxyethyl ethylenediamine, 2-hydroxyethyl propylenediamine, N,N-bis(hydroxyethyl)ethylenediamine, N,N-bis(2-hydroxypropyl)ethylenediamine, N,N-di-2-hydroxypropyl propylenediamine, N-methylethanolamine, diethanolamine, and chitosan.

These compounds can be used singly or in combination of two or more kinds thereof.

The most preferable examples of at least one active hydrogen group-containing compound selected from the group consisting of a polyol, a polyamine, or a compound having both hydroxyl and amino groups (b) used in the present invention are preferably selected from the above-described water-soluble polyols or water-soluble polyamines. Of these, particularly preferable examples include: bifunctional polyols such as ethylene glycol, diethylene glycol, propylene glycol, neopentyl glycol, 1,2-butanediol, 1,3-butanediol, 2,3-butanediol, and 1,4-butanediol; trifunctional polyols such as glycerin, trimethylolethane, and trimethylolpropane; tetrafunctional polyols such as pentaerythritol, erythritol, diglycerol, diglycerin, and ditrimethylolpropane; pentafunctional polyols such as arabitol; hexafunctional polyols such as dulcitol, sorbitol, mannitol, dipentaerythritol, and triglycerol; cyclic dextrin; and water-soluble polyamines such as ethylenediamine, propylenediamine, 1,4-diaminobutane, hexamethylenediamine, dipropylenetriamine, tris(2-aminoethyl)amine, diethylenetriamine, triethylenetetramine, and tetraethylenepentamine.

<Solvent for Dissolving At Least One Active Hydrogen Group-Containing Compound Selected from the Group Consisting of Polyol. Polyamine, and Compound Having both Hydroxyl and Amino Groups>

A solvent for dissolving at least one active hydrogen group-containing compound selected from a polyol, a polyamine, or a compound having both hydroxyl and amino groups used in the present invention is water, preferably ion-exchanged water. A hydrophilic solvent immiscible with the organic solvent can also be added as long as the effects of the present invention are not impaired.

In addition, for the purpose of further stabilizing the W/O emulsion, an additive can be added as long as the effects of the present invention are not impaired. Examples of such an additive include water-soluble salts such as sodium carbonate, calcium carbonate, potassium carbonate, sodium phosphate, potassium phosphate, calcium phosphate, sodium chloride, and potassium chloride. These additives can be used singly or in combination of two or more kinds thereof.

### <(C) Polyfunctional Isocyanate Compound Having At Least Two Isocyanate Groups>

The polyfunctional isocyanate compound used in the present invention can be used without any limitation as long as it is a polyfunctional isocyanate compound having at least two isocyanate groups. Among them, a compound having 2 to 6 isocyanate groups in the molecule is preferable, and a compound having 2 to 3 isocyanate groups in the molecule is more preferable.

Further, the component (c) can be (c2) a urethane prepolymer prepared by a reaction between a bifunctional isocyanate compound described later and a bifunctional polyol compound (hereinafter, also referred to as “component (c2)”). The (c2) urethane prepolymer corresponding to the isocyanate compound, which is generally used and contains an unreacted isocyanate group, can be used in the present invention without any limitation.

The component (c) can be broadly classified into, for example, aliphatic isocyanates, alicyclic isocyanates, aromatic isocyanates, other isocyanates, and urethane prepolymers (c2). Further, as the component (c), one kind of compound can be used, or a plurality of kinds of compounds can be used. When a plurality of kinds of compounds are used, the reference mass is the total amount of the plurality of kinds of compounds. Specific examples of these isocyanate compounds include the following monomers.

### (Aliphatic Isocyanate)

Bifunctional isocyanate monomers (corresponding to bifunctional polyisocyanate compounds constituting a urethane prepolymer) such as ethylene diisocyanate, trimethylene diisocyanate, tetramethylene diisocyanate, hexamethylene diisocyanate, octamethylene diisocyanate, nonamethylene diisocyanate, 2,2′-dimethylpentane diisocyanate, 2,2,4-trimethylhexamethylene diisocyanate, decamethylene diisocyanate, butene diisocyanate, 1,3-butadiene-1,4-diisocyanate, 2,4,4-trimethylhexamethylene diisocyanate, 1,6,11-trimethyl undecamethylene diisocyanate, 1,3,6-trimethylhexamethylene diisocyanate, 1,8-diisocyanate-4-isocyanate methyloctane, 2,5,7-trimethyl-1,8-diisocyanate-5-isocyanate methyloctane, bis(isocyanate ethyl)carbonate, bis(isocyanate ethyl)ether, 1,4-butylene glycol dipropylether-ω,ω′-diisocyanate, lysine diisocyanate methyl ester, and 2,4,4,-trimethylhexamethylene diisocyanate.

### (Alicyclic Isocyanate)

Bifunctional isocyanate monomers (corresponding to a bifunctional polyisocyanate compound constituting a urethane prepolymer) such as isophorone diisocyanate, (bicyclo[2.2.1]heptane-2,5-diyl)bismethylene diisocyanate, (bicyclo[2.2.1]heptane-2,6-diyl)bismethylene diisocyanate, 2β,5α-bis(isocyanate)norbornane, 2β,5β-bis(isocyanate)norbornane, 2β,6α-bis(isocyanate)norbornane, 2β,6β3-bis(isocyanate)norbornane, 2,6-di(isocyanate methyl)furan, 1,3-bis(isocyanate methyl)cyclohexane, dicyclohexylmethane-4,4′-diisocyanate, 4,4-isopropylidene bis(cyclohexylisocyanate), cyclohexane diisocyanate, methylcyclohexane diisocyanate, dicyclohexyl dimethylmethane diisocyanate, 2,2′-dimethyldicyclohexylmethane diisocyanate, bis(4-isocyanate-n-butylidene)pentaerythritol, diisocyanate dimerate, 2,5-bis(isocyanate methyl)-bicyclo[2,2,1]-heptane, 2,6-bis(isocyanate methyl)-bicyclo[2,2,1]-heptane, 3,8-bis(isocyanate methyl)tricyclodecane, 3,9-bis(isocyanate methyl)tricyclodecane, 4,8-bis(isocyanate methyl)tricyclodecane, 4,9-bis(isocyanate methyl)tricyclodecane, 1,5-diisocyanate decalin, 2,7-diisocyanate decalin, 1,4-diisocyanate decalin, 2,6-diisocyanate decalin, bicyclo [4.3.0]nonane-3,7-diisocyanate, bicyclo [4.3.0]nonane-4,8-diisocyanate, bicyclo[2.2.1]heptane-2,5-diisocyanate and bicyclo[2.2.1]heptane-2,6-diisocyanate, bicyclo[2,2,2]octane-2,5-diisocyanate, bicyclo[2,2,2]octane-2,6-diisocyanate, tricyclo[5,2,1,02,6]decane-3,8-diisocyanate, and tricyclo[5,2,1,02,6]decane-4,9-diisocyanate; and polyfunctional isocyanate monomers such as 2-isocyanate methyl-3-(3-isocyanate propyl)-5-isocyanate methyl-bicyclo[2,2,1]-heptane, 2-isocyanate methyl-3-(3-isocyanate propyl)-6-isocyanate methyl-bicyclo[2,2,1]-heptane, 2-isocyanate methyl-2-(3-isocyanate propyl)-5-isocyanate methyl-bicyclo[2,2,1]-heptane, 2-isocyanate methyl-2-(3-isocyanate propyl)-6-isocyanate methyl-bicyclo[2,2,1]-heptane, 2-isocyanate methyl-3-(3-isocyanate propyl)-5-(2-isocyanate ethyl)-bicyclo[2,2,1]-heptane, 2-isocyanate methyl-3-(3-isocyanate propyl)-6-(2-isocyanate ethyl)-bicyclo[2,1,1]-heptane, 2-isocyanate methyl-2-(3-isocyanate propyl)-5-(2-isocyanate ethyl)-bicyclo[2,2,1]-heptane, 2-isocyanate methyl-2-(3-isocyanate propyl)-6-(2-isocyanate ethyl)-bicyclo[2,2,1]-heptane, and 1,3,5-tris(isocyanate methyl)cyclohexane.

### (Aromatic Isocyanate)

Bifunctional isocyanate monomers (corresponding to a bifunctional polyisocyanate compound constituting a urethane prepolymer) such as xylylene diisocyanate (o-, m-,p-), tetrachloro-m-xylylene diisocyanate, methylene diphenyl-4,4′-diisocyanate, 4-chloro-m-xylylene diisocyanate, 4,5-dichloro-m-xylylene diisocyanate, 2,3,5,6-tetrabromo-p-xylylene diisocyanate, 4-methyl-m-xylylene diisocyanate, 4-ethyl-m-xylylene diisocyanate, bis(isocyanate ethyl)benzene, bis(isocyanate propyl)benzene, 1,3-bis(α,α-dimethylisocyanate methyl)benzene, 1,4-bis(α,α-dimethylisocyanate methyl)benzene, α,α,α′,α′-tetramethylxylylene diisocyanate, bis(isocyanate butyl)benzene, bis(isocyanate methyl)naphthalene, bis(isocyanate methyl)diphenylether, bis(isocyanate ethyl)phthalate, 2,6-di(isocyanate methyl)furan, phenylene diisocyanate (o-,m-,p-), tolylene diisocyanate, ethylphenylene diisocyanate, isopropylphenylene diisocyanate, dimethylphenylene diisocyanate, diethylphenylene diisocyanate, diisopropylphenylene diisocyanate, trimethylbenzene triisocyanate, benzene triisocyanate, 1,3,5-triisocyanate methylbenzene, 1,5-naphthalene diisocyanate, methylnaphthalene diisocyanate, biphenyldiisocyanate, 2,4-tolylene diisocyanate, 2,6-tolylene diisocyanate, 4,4′-diphenylmethane diisocyanate, 2,2′-diphenylmethane diisocyanate, 2,4′-diphenylmethane diisocyanate, 3,3′-dimethyl diphenylmethane-4,4′-diisocyanate, bibenzyl-4,4′-diisocyanate, bis(isocyanate phenyl)ethylene, 3,3′-dimethoxybiphenyl-4,4′-diisocyanate, phenylisocyanate methylisocyanate, phenylisocyanate ethylisocyanate, tetrahydronaphthylene diisocyanate, hexahydrobenzene diisocyanate, hexahydro diphenylmethane-4,4′-diisocyanate, diphenylether diisocyanate, ethylene glycol diphenylether diisocyanate, 1,3-propylene glycol diphenylether diisocyanate, benzophenone diisocyanate, diethylene glycol diphenylether diisocyanate, dibenzofuran diisocyanate, carbazole diisocyanate, ethylcarbazole diisocyanate, dichlorocarbazole diisocyanate, 2,4-tolylene diisocyanate, 2,6-tolylene diisocyanate; and polyfunctional isocyanate monomers such as mesitylene triisocyanate, triphenylmethanetriisocyanate, Polymeric MDI, naphthalenetriisocyanate, diphenylmethane-2,4,4′-triisocyanate, 3-methyl diphenylmethane-4,4′,6-triisocyanate, and 4-methyl-diphenylmethane-2,3,4′,5,6-pentaisocyanate.

### (Other Isocyanate)

Examples of other isocyanates include: a polyfunctional isocyanate comprising a diisocyanate such as hexamethylene diisocyanate as a main material and having the biuret structure, uretdione structure, or isocyanurate structure (for example, Japanese Unexamined Patent Publication No. 2004-534870 discloses a method for denaturing the biuret structure, uretdione structure, or isocyanurate structure of aliphatic polyisocyanate); and a polyfunctional compound as an adduct with a polyol such as trimethylolpropan (disclosed in references (e.g., “Polyurethane Resin Handbook” edited by Keiji Iwata, THE NIKKAN KOGYO SHIMBUN, LTD. (1987))).

((c2) Urethane Prepolymer)

In the present invention, (c2) a urethane prepolymer obtained by reacting a bifunctional isocyanate compound selected from a polyfunctional isocyanate compound having at least two isocyanate groups with a bifunctional polyol compound described below can be used.

Examples of the bifunctional polyol compound include the following.

### (Fatty Alcohol)

Bifunctional polyol monomers such as ethylene glycol, diethylene glycol, propylene glycol, dipropylene glycol, butylene glycol, 1,5-dihydroxypentane, 1,6-dihydroxyhexane, 1,7-dihydroxyheptane, 1,8-dihydroxyoctane, 1,9-dihydroxynonane, 1,10-dihydroxydecane, 1,11-dihydroxy undecane, 1,12-dihydroxydodecane, neopentylglycol, glyceryl monooleate, monoelaidin, polyethylene glycol, 3-methyl-1,5-dihydroxypentane, dihydroxyneopentyl, 2-ethyl-1,2-dihydroxyhexane, 2-methyl-1,3-dihydroxypropane, polyester polyol (a compound having hydroxyl groups only at both ends obtained by a condensation reaction between a polyol and a polybasic acid), polyether polyol (a compound obtained by ring-opening polymerization of an alkylene oxide or a reaction between a compound having two or more active hydrogen-containing groups in the molecule and an alkylene oxide and a denatured form thereof, which has hydroxyl groups only at both ends of the molecule), polycaprolactone polyol (a compound obtained by ring-opening polymerization of ε-caprolactone, which has hydroxyl groups only at both ends of the molecule), polycarbonate polyol (a compound obtained by phosgenating one or more kinds of low-molecular-weight polyols or a compound obtained by transesterification with ethylene carbonate, diethyl carbonate, diphenyl carbonate, or the like, which has hydroxyl groups only at both ends of the molecule), and polyacrylic polyol (a polyol compound obtained by polymerizing a (meth)acrylate acid ester or a vinyl monomer, which has hydroxyl groups only at both ends of the molecule).

### (Alicyclic Alcohol)

Bifunctional polyol monomers such as hydrogenated bisphenol A, cyclobutanediol, cyclopentanediol, cyclohexanediol, cycloheptane diol, cyclooctanediol, cyclohexanedimethanol, hydroxypropylcyclohexanol, tricyclo[5,2,1,02,6]decane-dimethanol, bicyclo[4,3,0]-nonanediol, dicyclohexanediol, tricyclo[5,3,1,13,9]dodecanediol, bicyclo[4,3,0]nonanedimethanol, tricyclo[5,3,1,13,9]dodecane-diethanol, hydroxypropyltricyclo[5,3,1,13,9]dodecanol, spiro[3,4]octanediol, butylcyclohexane diol, 1,1′-bicyclohexylidenediol, 1,4-cyclohexane dimethanol, 1,3-cyclohexane dimethanol, 1,2-cyclohexane dimethanol, and o-dihydroxyxylylene.

### (Aromatic Alcohol)

Bifunctional polyol monomers such as dihydroxynaphthalene, dihydroxybenzene, bisphenol A, bisphenol F, xylylene glycol, tetrabromobisphenol A, bis(4-hydroxyphenyl)methane, 1,1-bis(4-hydroxyphenyl)ethane, 1,2-bis(4-hydroxyphenyl)ethane, bis(4-hydroxyphenyl)phenylmethane, bis(4-hydroxyphenyl) diphenylmethane, bis(4-hydroxyphenyl)-1-naphthylmethane, 1,1-bis(4-hydroxyphenyl)-1-phenylethane, 2-(4-hydroxyphenyl)-2-(3-hydroxyphenyl)propane, 2,2-bis(4-hydroxyphenyl)butane, 1,1-bis(4-hydroxyphenyl)butane, 2,2-bis(4-hydroxyphenyl)-3-methylbutane, 2,2-bis(4-hydroxyphenyl)pentane, 3,3-bis(4-hydroxyphenyl)pentane, 2,2-bis(4-hydroxyphenyl)hexane, 2,2-bis(4-hydroxyphenyl)octane, 2,2-bis(4-hydroxyphenyl)-4-methylpentane, 2,2-bis(4-hydroxyphenyl)heptane, 4,4-bis(4-hydroxyphenyl)heptane, 2,2-bis(4-hydroxyphenyl)tridecane, 2,2-bis(4-hydroxyphenyl)octane, 2,2-bis(3-methyl-4-hydroxyphenyl)propane, 2,2-bis(3-ethyl-4-hydroxyphenyl)propane, 2,2-bis(3-n-propyl-4-hydroxyphenyl)propane, 2,2-bis(3-isopropyl-4-hydroxyphenyl)propane, 2,2-bis(3-sec-butyl-4-hydroxyphenyl)propane, 2,2-bis(3-tert-butyl-4-hydroxyphenyl)propane, 2,2-bis(3-cyclohexyl-4-hydroxyphenyl)propane, 2,2-bis(3-allyl-4′-hydroxyphenyl)propane, 2,2-bis(3-methoxy-4-hydroxyphenyl)propane, 2,2-bis(3,5-dimethyl-4-hydroxyphenyl)propane, 2,2-bis(2,3,5,6-tetramethyl-4-hydroxyphenyl)propane, bis(4-hydroxyphenyl)cyanomethane, 1-cyano-3,3-bis(4-hydroxyphenyl)butane, 2,2-bis(4-hydroxyphenyl)hexafluoropropane, 1,1-bis(4-hydroxyphenyl)cyclopentane, 1,1-bis(4-hydroxyphenyl)cyclohexane, 1,1-bis(4-hydroxyphenyl)cycloheptane, 1,1-bis(3-methyl-4-hydroxyphenyl)cyclohexane, 1,1-bis(3,5-dimethyl-4-hydroxyphenyl)cyclohexane, 1,1-bis(3,5-dichloro-4-hydroxyphenyl)cyclohexane, 1,1-bis(3-methyl-4-hydroxyphenyl)-4-methylcyclohexane, 1,1-bis(4-hydroxyphenyl)-3,3,5-trimethylcyclohexane, 2,2-bis(4-hydroxyphenyl)norbornane, 2,2-bis(4-hydroxyphenyl)adamantane, 4,4′-dihydroxy diphenylether, 4,4′-dihydroxy-3,3′-dimethyl diphenylether, ethylene glycol bis(4-hydroxyphenyl)ether, 4,4′-dihydroxy diphenylsulfide, 3,3′-dimethyl-4,4′-dihydroxy diphenylsulfide, 3,3′-dicyclohexyl-4,4′-dihydroxy diphenylsulfide, 3,3′-diphenyl-4,4′-dihydroxy diphenylsulfide, 4,4′-dihydroxy diphenylsulfoxide, 3,3′-dimethyl-4,4′-dihydroxy diphenylsulfoxide, 4,4′-dihydroxy diphenylsulfone, 4,4′-dihydroxy-3,3′-dimethyl diphenylsulfone, bis(4-hydroxyphenyl)ketone, bis(4-hydroxy-3-methylphenyl)ketone, 7,7′-dihydroxy-3,3′,4,4′-tetrahydro-4,4,4′,4′-tetramethyl-2,2′-spirobi(2H-1-benzopyran), trans-2,3-bis(4-hydroxyphenyl)-2-butene, 9,9-bis(4-hydroxyphenyl)fluorene, 3,3-bis(4-hydroxyphenyl)-2-butanone, 1,6-bis(4-hydroxyphenyl)-1,6-hexanedione, 4,4′-dihydroxybiphenyl, m-dihydroxyxylylene, p-dihydroxyxylylene, 1,4-bis(2-hydroxyethyl)benzene, 1,4-bis(3-hydroxypropyl)benzene, 1,4-bis(4-hydroxybutyl)benzene, 1,4-bis(5-hydroxypentyl)benzene, 1,4-bis(6-hydroxyhexyl)benzene, 2,2-bis[4-(2″-hydroxyethyloxy)phenyl]propane, hydroquinone, and resorcinol.

### (Method for Producing (c2) Urethane Prepolymer)

The (c2) urethane prepolymer can be produced by reacting the bifunctional isocyanate group and the bifunctional polyol compound described above. However, in the present invention, in the (c2) urethane prepolymer, both ends of the molecule must be isocyanate groups. The method for producing the (c2) urethane prepolymer having an isocyanate group at both ends is not particularly limited, and a known method can be used. For example, a method in which the (c2) urethane prepolymer is produced in a range that the number of moles of isocyanate groups (n5) in the bifunctional isocyanate group-containing monomer and the number of moles of groups having active hydrogen of the bifunctional polyol (n6) satisfy 1<(n5)/(n6)≤2.3. In a case in which two or more kinds of bifunctional isocyanate group-containing monomers are used, the number of moles of the isocyanate groups (n5) is the total number of moles of isocyanate groups of the bifunctional isocyanate group-containing monomers. In a case in which two or more kinds of bifunctional polyols are used, the number of moles of the group having the active hydrogen (n6) is the total number of moles of active hydrogen of the bifunctional polyols.

Although not particularly limited, the (c2) urethane prepolymer has an isocyanate equivalent (a value obtained by dividing the molecular weight of the (c2) urethane prepolymer by the number of isocyanate groups in one molecule) is preferably from 300 to 5000, more preferably from 500 to 3000, particularly preferably from 700 to 2000. The (c2) urethane prepolymer in the present invention is preferably a linear polymer synthesized from a bifunctional isocyanate group-containing monomer and a bifunctional polyol. In that case the number of isocyanate groups in one molecule is 2.

The isocyanate equivalent of the (c2) urethane prepolymer can be determined by quantifying isocyanate groups of the (c2) urethane prepolymer in accordance with JIS K7301. The isocyanate groups can be quantified by the following back titration method. First, the obtained (c2) urethane prepolymer is dissolved in a dry solvent. Next, di-n-butylamine, which is clearly in excess of the amount of isocyanate groups contained in the (c2) urethane prepolymer and has a known concentration, is added to the dry solvent, thereby react all isocyanate groups of the (c2) urethane prepolymer with di-n-butylamine. Subsequently, the unconsumed (not involved in the reaction) di-n-butylamine is then titrated with an acid to determine the amount of di-n-butylamine consumed. Since the consumed di-n-butylamine and the isocyanate groups of the (c2) urethane prepolymer have the same amount, the isocyanate equivalent can be determined. Further, since the (c2) urethane prepolymer is a linear urethane prepolymer having isocyanate groups at both ends, the number average molecular weight of the (c2) urethane prepolymer is twice the isocyanate equivalent. The molecular weight of this (c2) urethane prepolymer tends to match the value measured by gel permeation chromatography (GPC). In a case in which the (c2) urethane prepolymer and the bifunctional isocyanate group-containing monomer are used in combination, a mixture of both can be measured according to the above-described method.

Furthermore, the isocyanate content ((I): molality (mol/kg)) of the (c2) urethane prepolymer and the urethane bond content ((U): molality (mol/kg)) present in the urethane prepolymer (c2) is preferably 1≤(U)/(I)≤10. This range is the same also when the urethane prepolymer (c2) and the bifunctional isocyanate group-containing monomer are used in combination.

The isocyanate content ((I): molality (mol/kg)) is the value obtained by multiplying the reciprocal of the isocyanate equivalent by 1000. Further, the urethane bond content ((U): molality (mol/kg)) present in the urethane prepolymer can be obtained as a theoretical value by the following method. In other words, given that the contents of a bifunctional polyol constituting the (c2) urethane prepolymer and unreacted isocyanate groups present in the bifunctional isocyanate group-containing monomer correspond to the total isocyanate content ((aI): molality (mol/kg)), the urethane bond content ((U): molality (mol/kg)) is the value ((U)=(aI)−(I)) obtained by subtracting the isocyanate content ((I): molality (mol/kg)) from the content of all isocyanate groups in the component (B) ((aI): molality (mol/kg)).

Further, in the reaction of the (c2) urethane prepolymer, it is also possible to perform heating or add an urethanization catalyst as needed. Any suitable urethanization catalyst can be used, and a specific example can be an urethanization catalyst described later.

The most preferable examples of the polyfunctional isocyanate compound having at least two isocyanate groups (c) used in the present invention can be mentioned. From the viewpoint of controlling the strength and reactivity of the formed microballoons, alicyclic isocyanate selected from isophorone diisocyanate, 1,3-bis(isocyanate methyl)cyclohexane, or (bicyclo[2.2.1]heptane-2,5(2,6)-diyl)bismethylene diisocyanate, aromatic isocyanate selected from 2,4-tolylene diisocyanate, 2,6-tolylene diisocyanate, 4,4′-diphenylmethane diisocyanate, or xylylene diisocyanate (o-, m-,p-), and a polyfunctional isocyanate comprising a diisocyanate such as hexamethylene diisocyanate or tolylene diisocyanate as a main material and having the biuret structure, uretdione structure, or isocyanurate structure can be mentioned. Examples of an adduct with a trifunctional or higher functional polyol include a polyfunctional isocyanate and (B12) a urethane prepolymer.

<Monofunctional Active Hydrogen Compound Containing Only One Active Hydrogen Group Selected from Amino Group and Hydroxyl Group (d)>

In the present invention, as the monofunctional active hydrogen compound containing only one active hydrogen group selected from an amino group and a hydroxyl group, a known compound can be used without particular limitation.

Examples thereof include monofunctional alcohol, mono-substituted polyalkylene glycol ether, polyalkylene glycol monoester such as a lower or higher fatty acid-ethylene oxide condensate, and monofunctional amine. The following are examples of specific examples thereof.

### (Monofunctional Alcohol)

Methylalcohol, ethylalcohol, n-propylalcohol, isopropylalcohol, n-butylalcohol, isobutylalcohol, t-butylalcohol, 1-pentylalcohol, 1-hexylalcohol, 1-heptylalcohol, 3-methyl-1-hexylalcohol, 4-methyl-1-hexylalcohol, 2-ethyl-1-hexylalcohol, 5-methyl-1-heptylalcohol, 1-octylalcohol, 1-nonanol, 1-decanol, 3,7-dimethyl-1-octanol, 1-dodecanol, 1-undecanol, 1-tridecanol, 3,3,5-trimethyl-1-hexanol, 1-tetradecanol, I-pentadecanol, 1-hexadecanol, 1-heptadecanol, I-octadecanol, 1-eicosanol, 1-docosanol, and 1-tricosanol.

### (Mono-Substituted Polyalkylene Glycol Ether)

2-Methoxy methanol, diethylglycol monomethylether, triethylene glycol monomethylether, tetraethylene glycol monomethylether, pentaethylene glycol monomethylether, hexaethylene glycol monomethylether, heptaethylene glycol monomethylether, octaethylene glycol monomethylether, nonaethylene glycol monomethylether, decaethylene glycol monomethylether, dodecaethylene glycol monomethylether, 1-methoxy-2-propanol, 1-methoxy-2-propanol, 1-isopropyl-2-propanol, 1-methoxy-2-butanol, 1,3-diethoxypropanol, polyethylene glycol monooleyl ether, and polyoxyethylene lauryl ether.

(Polyalkylene Glycol Monoester such as Lower or Higher Fatty Acid-Ethylene Oxide Condensate)

Polyethylene glycol monolaurate and polyethylene glycol monostearate.

### (Monofunctional Amine)

Ethylamine, n-propylamine, isopropylamine, n-butylamine, isobutylamine, n-pentylamine, isopentylamine, n-hexylamine, cyclohexylamine, n-heptylamine, n-octylamine, 2-ethylhexylamine, n-nonylamine, n-decylamine, n-dodecylamine, n-tetradecylamine, n-hexadecylamine, n-octadecylamine, benzylamine, and phenethylamine.

The molecular weight of the (d) monofunctional active hydrogen compound containing only one active hydrogen group selected from an amino group and a hydroxyl group used in the present invention is not particularly limited. In a case in which microballoons obtained by the method of the present invention are blended with a resin, for example, in a case in which the microballoons are blended with a urethane resin for foaming of the urethane resin, considering the dispersion in the urethane resin, the molecular weight of the (d) monofunctional active hydrogen compound containing only one active hydrogen group selected from an amino group and a hydroxyl group is preferably 130 or less.

Specific examples of (d) a monofunctional active hydrogen compound containing only one active hydrogen group selected from an amino group and a hydroxyl group which has a molecular weight of 130 or less include the following, and these can be used singly or in combination of two or more kinds thereof.

(Monofunctional Active Hydrogen Compound Containing Only One Hydroxyl Group and Having Molecular Weight of 130 or less)

Methylalcohol, ethylalcohol, n-propylalcohol, isopropylalcohol, n-butylalcohol, isobutylalcohol, t-butylalcohol, 1-pentylalcohol, 1-hexylalcohol, I-heptylalcohol, 2-methoxy methanol, and diethylene glycol monomethylether.

(Monofunctional Active Hydrogen Compound Containing Only One Amino Group and Having Molecular Weight of 130 or less)

Ethylamine, n-propylamine, isopropylamine, n-butylamine, isobutylamine, n-pentylamine, isopentylamine, n-hexylamine, cyclohexylamine, n-heptylamine, n-octylamine, and 2-ethylhexylamine.

Of these, in the present invention, a monofunctional active hydrogen compound containing only one hydroxyl group and having a molecular weight of 130 or less is preferably used.

### <Urethanization Catalyst>

Any suitable urethanization catalyst can be used in the present invention. Specific examples thereof include triethylenediamine, hexamethylenetetramine, N,N-dimethyloctylamine, N,N,N′,N′-tetramethyl-1,6-diaminohexane, 4,4′-trimethylene bis(1-methylpiperidine), 1,8-diazabicyclo-(5,4,0)-7-undecene, dimethyltin dichloride, dimethyltin bis(isooctylthioglycolate), dibutyltin dichloride, dibutyltin dilaurate, dibutyltin maleate, dibutyltin maleate polymer, dibutyltin diricinolate, dibutyltin bis(dodecylmercaptide), dibutyltin bis(isooctylthioglycolate), dioctyltin dichloride, dioctyltin maleate, dioctyltin maleate polymer, dioctyltin bis(butylmaleate), dioctyltin dilaurate, dioctyltin diricinolate, dioctyltin dioleate, dioctyltin di(6-hydroxy)caproate, dioctyltin bis(isooctylthioglycolate), didodecyltin diricinolate, and various metal salts such as, for example, copper oleate, copper acetylacetone, iron acetylacetone, iron naphthenate, iron lactate, iron citrate, iron gluconate, potassium octoate, and 2-ethylhexyl titanate.

### EXAMPLES

Next, the present invention will be described in detail with reference to Examples and Comparative Examples, but the present invention is not limited to Examples. In the following Examples and Comparative Examples, the evaluation methods and the like for each of the following components and the urethane resin are as follows.

### Component (a): Surfactant

Sorbitan monostearate
Sorbitan monooleate
   Component (a): Organic solvent
n-Hexane
Corn oil
   Component (b): a polyol, a polyamine, or a compound having both hydroxyl and amino groups
Tris(2-aminoethyl)amine
   (c) Polyfunctional isocyanate compound having at least two isocyanate groups
Hexamethylene diisocyanate
2,4-Tolylene diisocyanate
   (d) Monofunctional active hydrogen compound containing only one active hydrogen group selected from an amino group and a hydroxyl group
Methyl alcohol (molecular weight: 32)
1-Eicosanol (molecular weight: 298)
Hexylamine (molecular weight: 101.19)
10-amino-1-decanol (molecular weight: 173.3)

### Example 1

### Method for Producing Microballoons 1

The component (a) was prepared by adding 5 parts by mass of sorbitan monostearate to 100 parts by mass of n-hexane and dissolving the mixture. Next, the component (b) was prepared by dissolving 5 parts by mass of tris(2-aminoethyl)amine in 50 parts by mass of water. Then, the prepared components (a) and (b) were mixed and stirred using a high-speed shear-disperser at 2000 rpm for 15 minutes at 25° C., thereby preparing a W/O emulsion. To the prepared W/O emulsion, 9 parts by mass of hexamethylene diisocyanate dissolved in 17 parts by mass of n-hexane was added dropwise at 25° C. After the dropwise addition, the reaction was carried out at 60° C. for 1 hour with stirring, thereby obtaining a microballoon dispersion liquid consisting of polyurea. The microballoons were taken out from the obtained microballoon dispersion liquid by filter paper filtration, the recovered microballoons were dispersed in 50 parts by mass of methyl alcohol and stirred at 25° C. for 12 hours, the microballoons were taken out again by filter paper filtration and dried in a circulation dryer at 60° C. for 12 hours, thereby obtaining microballoons 1.

The obtained microballoons 1 had an average primary particle diameter of about 40 μm and had excellent dispersibility. The primary particles did not aggregate with each other.

### Example 2

Method for Producing Microballoons 2 To a microballoon dispersion liquid obtained in the same manner as in Example 1, 50 parts by mass of methyl alcohol was added dropwise, and the mixture was stirred at 25° C. for 12 hours, the microballoons were taken out by filter paper filtration, and dried in a circulation dryer at 60° C. for 12 hours, thereby obtaining microballoons 2.

The obtained microballoons 2 had an average primary particle diameter of about 40 μm and had excellent dispersibility. The primary particles did not aggregate with each other.

### Example 3

### Method for Producing Microballoons 3

To a microballoon dispersion liquid obtained in the same manner as in Example 1, 50 parts by mass of 1-eicosanol was added dropwise, and the mixture was stirred at 25° C. for 12 hours, the microballoons were taken out by filter paper filtration, and dried in a circulation dryer at 60° C. for 12 hours, thereby obtaining microballoons 3.

The obtained microballoons 3 had an average primary particle diameter of about 40 μm and had excellent dispersibility. The primary particles did not aggregate with each other.

### Comparative Example 1

### Method for Producing Microballoons 4

The microballoons were taken out by filter paper filtration from a microballoon dispersion liquid obtained in the same manner as in Example 1 and dried in a circulation dryer at 60° C. for 12 hours, thereby obtaining microballoons 4.

The obtained microballoons 4 aggregated, and therefore the primary particle diameter could not be measured.

### Comparative Example 2

### Method for Producing Microballoons 5

To a microballoon dispersion liquid obtained in the same manner as in Example 1, 50 parts by mass of ethylene glycol was added dropwise, and the mixture was stirred at 25° C. for 12 hours, the microballoons were taken out by filter paper filtration, and dried in a circulation dryer at 60° C. for 12 hours, thereby obtaining microballoons 5.

The obtained microballoons 5 aggregated, and therefore the primary particle diameter could not be measured.

### Example 4

Using the microballoons 1 obtained in Example 1, a urethane resin for a polishing pad was prepared according to the following formulation.

First, a terminal isocyanate urethane prepolymer (Pre-1) was prepared according to the following formulation.

### <Method for Producing Terminal Isocyanate Urethane Prepolymer (Pre-1)>

In a nitrogen atmosphere, 1000 g of 2,4-tolylene diisocyanate and 1800 g of polyoxytetramethylene glycol (number average molecular weight: 1000) were reacted in a flask equipped with a nitrogen inlet tube, a thermometer, and stirrer at 70° C. for 4 hours. Then, 240 g of diethylene glycol was added and further reacted at 70° C. for 4 hours, thereby obtaining a terminal isocyanate urethane prepolymer having an isocyanate equivalent of 905 (Pre-1 was obtained).

Next, polyrotaxane (RX-1) to be used for a curing agent was obtained by the following formulation. The method for producing polyrotaxane was acquired according to the method described in WO2018/092826.

### <Method for Producing Polyrotaxane Monomer (RX-1) Used>

As a polymer for forming an axle molecule, 10 g of linear polyethylene glycol having a molecular weight of 10,000, 100 mg of 2,2,6,6-tetramethyl-1-piperidinyloxy radical and 1 g of sodium bromide were prepared, and each component was dissolved in 100 mL of water. To this solution, 5 mL of a commercially available sodium hypochlorite aqueous solution (effective chlorine concentration: 5%) was added, and the mixture was stirred at room temperature for 10 minutes. Then, ethanol was added in a range of up to 5 mL to terminate the reaction. Then, after extraction with 50 mL of methylene chloride, methylene chloride was distilled off. The obtained product was dissolved in 250 mL of ethanol, and then reprecipitated at a temperature of −4° C. for 12 hours to recover the solid, and the solid was dried.

Thereafter, 3 g of the obtained solid and 12 g of α-cyclodextrin were each dissolved in 50 mL of warm water at 70° C., and each of the obtained solutions was mixed and shaken well. The mixed solution was then reprecipitated at a temperature of 4° C. for 12 hours, and the precipitated inclusion complex was lyophilized and recovered. Then, 0.13 g of adamantanamine was dissolved in 50 mL of dimethylformamide at room temperature, the above inclusion complex was added, and the mixture was immediately well shaken well. Subsequently, 0.38 g of benzotriazole-1-yl-oxy-tris(dimethylamino) phosphonium hexafluorophosphate was further added and shaken well. Further, 0.14 mL of diisopropylethylamine was added and shaken well, thereby obtaining a slurry-like reagent.

The obtained slurry-like reagent was allowed to stand at 4° C. for 12 hours. Then, 50 mL of a dimethylformamide/methanol mixed solvent (volume ratio: 1/1) was added, mixed, and centrifuged, and the supernatant was discarded. Further, after washing with the above-described dimethylformamide/methanol mixed solution, washing with methanol and centrifugation were performed, thereby obtaining a precipitate. The obtained precipitate was dried by vacuum drying and then dissolved in 50 mL of dimethylsulfoxide, and the obtained transparent solution was added dropwise to 700 mL of water, thereby precipitating polyrotaxane. The precipitated polyrotaxane was collected by centrifugation and dried under vacuum. It was further dissolved in dimethylsulfoxide, precipitated in water, recovered, and dried, thereby obtaining purified polyrotaxane. The purified polyrotaxane was dissolved in an amount of 500 mg in 50 mL of a 1 mol/L NaOH aqueous solution, 3.83 g (66 mmol) of propylene oxide was added, and the mixture was stirred at room temperature for 12 hours under an argon atmosphere. Then, the polyrotaxane solution was neutralized to a pH of 7 to 8 using a 1 mol/L HCl aqueous solution, dialyzed using a dialysis tube, and then freeze-dried, thereby obtaining hydroxypropylated polyrotaxane. The obtained hydroxypropylated polyrotaxane was identified by 1H-NMR and GPC, and thus it was confirmed to be a hydroxypropylated polyrotaxane having a desired structure. A mixed solution was prepared by dissolving 5 g of the obtained hydroxypropylated polyrotaxane in 15 g of ε-caprolactone at 80° C. This mixed solution was stirred at 110° C. for 1 hour while blowing dry nitrogen, 0.16 g of a 50 wt % xylene solution of tin (11) 2-ethylhexanoate was added, and the mixture was stirred at 130° C. for 6 hours. Then, xylene was added, thereby obtaining a solution of polycaprolactone-modified polyrotaxane xylene into which a side chain was introduced, with a non-volatile concentration of about 35% by mass. The obtained polycaprolactone-modified polyrotaxane xylene solution was added dropwise to hexane, recovered, and dried, thereby obtaining a side chain-modified polyrotaxane (RX-1) having an OH group as the end of the side chain.

The physical properties of this polyrotaxane (A) (RX-1) were as follows.

Weight average molecular weight (Mw) (GPC): 200,000

Hydroxyl value: 87 mg KOFI/g

Degree of modification of side chain: 0.5 (50% when expressed in %)

Molecular weight of side chain: About 350 on average

### <Method for Producing Urethane Resin for Polishing Pad>

A uniform solution was formed by mixing 24 parts by mass of RX-1 produced above and 5 parts by mass of 44′-methylene bis(o-chloroaniline) (MOCA) at 120° C., and then sufficiently degassed, thereby preparing liquid A. Separately, 21 parts by mass of the microballoons 1 obtained in Example 1 were added to the 71 parts by mass of Pre-1 produced above, which was heated to 70° C., and stirred with a planetary centrifugal mixer, thereby obtaining a uniform solution. The liquid A adjusted to 100° C. was added thereto, and the mixture was stirred with the planetary centrifugal mixer, thereby obtaining a uniform polymerizable composition. The polymerizable composition was poured into a mold and cured at 100° C. for 15 hours, thereby obtaining a urethane resin.

### <Method for Producing Polishing Pad>

The obtained urethane resin was sliced, thereby obtaining a polishing pad consisting of a urethane resin having a thickness of 1 mm.

The polishing rate of the urethane resin obtained above was 3.3 μm/hr, and the scratch resistance was 1. Each evaluation method is shown below.

(1) Polishing rate: Polishing conditions are shown below. 30 wafers were used.

The polishing rate when polishing was performed was measured under the following conditions. The polishing rate is an average value of 50 wafers.

Polishing pad: A pad with a size of 380 mmϕ and a thickness of 1 mm with concentric grooves formed on the surface.

Object to be polished: 2-inch sapphire wafer

Slurry: FUJIMI COMPOL-80 undiluted solution

Pressure: 411 g/cm²

Rotation speed: 60 rpm

Time: 1 hour

(2) Scratch resistance: It was confirmed whether or not 50 wafers were scratched when polished under the conditions described in (1) above. The evaluation was carried out according to the following criteria.

1: No scratches can be confirmed on all 50 wafers.

2: Of the 50 wafers, scratches can be confirmed on one or two wafers.

3: Of the 50 wafers, scratches can be confirmed on three or four wafers.

4: Of the 50 wafers, scratches can be confirmed on five to ten wafers.

### Examples 5 and 6, Comparative Examples 3 and 4

Polishing pads each consisting of a urethane resin were prepared and evaluated by the same method as in Example 4 except that the curable compositions having the compositions shown in Table 1 were used. The results are shown in Table 1.

### Example 7

### Method for Producing Microballoons 6

The component (a) was prepared by adding 10 parts by mass of sorbitan monooleate to 100 parts by mass of corn oil and dissolving the mixture. Next, the component (b) was prepared by dissolving 10 parts by mass of tris(2-aminoethyl)amine in 50 parts by mass of water. Then, the prepared components (a) and (b) were mixed and stirred using a high-speed shear-disperser at 1500 rpm for 15 minutes at 25° C., thereby preparing a W/O emulsion. To the prepared W/O emulsion, 11.9 parts by mass of 2,4-tolylene diisocyanate dissolved in 36 parts by mass of corn oil was added dropwise at 25° C. After the dropwise addition, the reaction was carried out at 60° C. for 1 hour with stirring, thereby obtaining a microballoon dispersion liquid consisting of polyurea. The microballoons were taken out from the obtained microballoon dispersion liquid by filter paper filtration, the recovered microballoons were dispersed in 50 parts by mass of methyl alcohol and stirred at 25° C. for 12 hours, the microballoons were taken out again by filter paper filtration and dried in a circulation dryer at 60° C. for 12 hours, thereby obtaining microballoons 6.

The obtained microballoons 6 had an average primary particle diameter of about 30 μm and had excellent dispersibility. The primary particles did not aggregate with each other.

### Example 8

### Method for Producing Microballoons 7

To the microballoon dispersion liquid obtained in the same manner as in Example 1, 5 parts by mass of methyl alcohol was added dropwise and stirred at 60° C. for 1 hour. The microballoons were taken out by filter paper filtration and dried in a circulation dryer at 60° C. for 12 hours, thereby obtaining microballoons 7.

The obtained microballoons 7 had an average primary particle diameter of about 30 μm and had excellent dispersibility. The primary particles did not aggregate with each other.

### Example 9

### Method for Producing Microballoons 8

To the microballoon dispersion liquid obtained in the same manner as in Example 8, 2.3 parts by mass of hexylamine was added dropwise and stirred at 60° C. for 1 hour. The microballoons were taken out by filter paper filtration and dried in a circulation dryer at 60° C. for 12 hours, thereby obtaining microballoons 8.

The obtained microballoons 8 had an average primary particle diameter of about 30 μm and had excellent dispersibility. The primary particles did not aggregate with each other.

### Comparative Example 5

### Method for Producing Microballoons 9

To the microballoon dispersion liquid obtained in the same manner as in Example 8, 3.7 parts by mass of 10-amino-1-decanol was added dropwise and stirred at 60° C. for 1 hour. The microballoons were taken out by filter paper filtration and dried in a circulation dryer at 60° C. for 12 hours, thereby obtaining microballoons 9.

The obtained microballoons 9 aggregated, and therefore the primary particle diameter could not be measured.

Examples 10 to 12, Comparative Example 6 Polishing pads each consisting of a urethane resin were prepared and evaluated by the same method as in Example 4 except that the curable compositions having the compositions shown in Table 1 were used. The results are shown in Table 1.

TABLE 1

| | | Urethane resin | Polishing | |
|---|---|---|---|---|
| | Microballoons | composition | rate | Scratch |
| | (parts by mass) | (parts by mass) | μm/hr | resistance |
| Example 4 | Microballoons | RX-1 (24)/Pre-1 | 3.3 | 1 |
| | 1 (21) | (71)/MOCA (5) | | |
| Example 5 | Microballoons | RX-1 (24)/Pre-1 | 3.2 | 1 |
| | 2 (21) | (71)/MOCA (5) | | |
| Example 6 | Microballoons | RX-1 (24)/Pre-1 | 2.8 | 1 |
| | 3 (21) | (71)/MOCA (5) | | |
| Comparative | Microballoons | RX-1 (24)/Pre-1 | 2.0 | 4 |
| Example 3 | 4 (21) | (71)/MOCA (5) | | |
| Comparative | Microballoons | RX-1 (24)/Pre-1 | 2.1 | 3 |
| Example 4 | 5 (21) | (71)/MOCA (5) | | |
| Example 10 | Microballoons | RX-1 (24)/Pre-1 | 3.8 | 1 |
| | 6 (21) | (71)/MOCA (5) | | |
| Example 11 | Microballoons | RX-1 (24)/Pre-1 | 3.7 | 1 |
| | 7 (21) | (71)/MOCA (5) | | |
| Example 12 | Microballoons | RX-1 (24)/Pre-1 | 3.7 | 1 |
| | 8 (21) | (71)/MOCA (5) | | |
| Comparative | Microballoons | RX-1 (24)/Pre-1 | 2.5 | 2 |
| Example 6 | 9 (21) | (71)/MOCA (5) | | |

As can be seen from the results in Table 1, the microballoons having excellent dispersibility obtained by the production method of the present invention can be uniformly dispersed in the urethane resin, and as a result, the polishing rate and scratch resistance are favorable. Meanwhile, in microballoons with poor dispersibility as in the Comparative Examples, the polishing rate and scratch resistance are reduced due to factors such as the occurrence of local changes in hardness and density in the urethane resin that causes uneven polishing.

1. A method for producing microballoons, comprising:
mixing and stirring (a) an organic solvent solution containing surfactant and (b) an aqueous solution containing at least one active hydrogen group-containing compound selected from the group consisting of a polyol, a polyamine, and a compound having both hydroxyl and amino groups so as to preparing a water-in-oil (W/O) emulsion in which the organic solvent solution is a continuous phase and the aqueous solution is a dispersed phase;
adding (c) a polyfunctional isocyanate compound having at least two isocyanate groups to the water-in-oil (W/O) emulsion to react the polyfunctional isocyanate compound and the active hydrogen group-containing compound on the interface of the water-in-oil (W/O) emulsion to form microballoons consisting of a polyurethane (urea) so as to obtain a microballoon dispersion liquid in which the formed microballoons are dispersed; and
treating the microballoons in a solution containing (d) a monofunctional active hydrogen compound containing only one active hydrogen group selected from an amino group and a hydroxyl group after forming the microballoons.

2. The method for producing microballoons according to claim 1, wherein the microballoons are treated in (d) a solution containing a monofunctional active hydrogen compound containing only one active hydrogen group selected from an amino group and a hydroxyl group such that the microballoons are once separated from the microballoon dispersion liquid, the separated microballoons are dispersed in the solution containing (d) a monofunctional active hydrogen compound containing only one active hydrogen group selected from an amino group and a hydroxyl group, and then the microballoons are re-separated.

3. The method for producing microballoons according to claim 1, wherein the microballoons are treated in a solution containing (d) a monofunctional active hydrogen compound containing only one active hydrogen group selected from an amino group and a hydroxyl group such that the microballoon dispersion liquid and the solution containing (d) a monofunctional active hydrogen compound containing only one active hydrogen group selected from an amino group and a hydroxyl group are mixed, and then the microballoons are separated from the microballoon dispersion liquid.

4. The method for producing microballoons according to claim 1, wherein a molecular weight of (d) the monofunctional active hydrogen compound containing only one active hydrogen group selected from an amino group and a hydroxyl group is 130 or less.

5. The method for producing microballoons according to claim 2, wherein a molecular weight of (d) the monofunctional active hydrogen compound containing only one active hydrogen group selected from an amino group and a hydroxyl group is 130 or less.

6. The method for producing microballoons according to claim 3, wherein a molecular weight of (d) the monofunctional active hydrogen compound containing only one active hydrogen group selected from an amino group and a hydroxyl group is 130 or less.
